(19) United States
(12) Patent Application Publication
(10) Pub. No.: US 2023/0402144 A1
(43) Pub. Date: Dec. 14, 2023

(54) METHODS AND SYSTEMS FOR PREDICTIVE MODELING

(71) Applicant: McKesson Corporation, Irving, TX (US)

(72) Inventors: Yvonne Vasetta Edmonds, Irving, TX (US); Michael James Ziemke, Keller, TX (US)

(21) Appl. No.: 17/839,224

(22) Filed: Jun. 13, 2022

(51) Int. Cl. G16H 20/10; G06F 40/20

(52) U.S. Cl. CPC G16H 20/10; G06F 40/20

(57) ABSTRACT

Methods, systems, and apparatuses for predicting prescription drug products or substitution drug products for inclusion in a company's portfolio.

### BACKGROUND

The vast majority of US prescriptions are FDA-approved generic drugs. Several drug manufacturers may produce a version of the same generic drug. Generic drugs are affordable, and have the same high quality, strength, purity, and stability as brand-name drugs. However, differences in product attributes, including pill color, pill shape, flavor, inactive ingredients, etc., may lead to patient preference for the version of a generic drug supplied by a specific manufacturer. In cases where preferred supplier's, or manufacturer's, product is not available, due to a supply disruption or an unforeseen increase in demand, an equivalent item may be substituted for the preferred item. However, at the time of ordering the product, some customers may provide a “no substitution” election with respect to his/her preferred product. This “no substitution” data provides unique insight into large scale patient preference data at the transactional level. The ability to predict patient preference based on product attributes can help determine which products should be included in a portfolio of products and help predict adoption of new products. Currently, only in-house transaction data is used to understand pharmacy and patient preference and institutional/expert knowledge from pharmacists. However, transaction data does not provide insight into the underlying factors that drive patient preference. In addition, since transaction data is only available for generic products sold by a company, the current solution provides limited insight into preferences for, or adoption of, new product prior to inclusion in a company's portfolio.

Thus, drug suppliers can greatly benefit from the use of a machine learning methods and models for the prediction of consumer drug preference in order to enhance a drug supplier's ability to predict products for inclusion into the drug supplier's portfolio and predict the adoption of new products. However, one of the biggest issues facing the use of machine learning is the lack of availability of large, annotated datasets.

The annotation of data is not only expensive and time consuming but also highly dependent on the availability of expert observers. The limited amount of training data can inhibit the performance of supervised machine learning algorithms which often need very large quantities of data on which to train to avoid overfitting. So far, much effort has been directed at extracting as much information as possible from what data is available. One area in particular that suffers from lack of large, annotated datasets is the analysis of consumer preference data associated with prescription drugs. The ability to analyze the consume preference data associated with prescription drugs is critical to managing a supplier's portfolio of both prescription drugs and substitution, or generic, drugs. However, in many instances, insufficient data are available to train machine learning algorithms to accurately predict consumer preferences. Natural language processing techniques (NLP), product attribute data, and substitution transaction data may be utilized to assist in identifying products prior to their inclusion in a company's portfolio. A patient preference prediction may enhance a company's ability to make strategic portfolio decisions that benefit both pharmacy customers and patients.

Thus, there is a need for improved systems and methods for determining and utilizing consumer preference data sets and drug feature data sets for use in machine learning applications. Therefore, it is an object of the invention to provide computer-implemented systems and methods that have improved capability to determine and utilize consumer preference data sets and drug feature data sets for training machine learning applications to make predictions, including predicting products for inclusion in a company's portfolio.

### SUMMARY

It is to be understood that both the following general description and the following detailed description are exemplary and explanatory only and are not restrictive.

In an embodiment, disclosed are methods comprising receiving, by a computing device, consumer preference data associated with a prescription drug, determining drug feature data associated with a plurality of drugs, generating, based on the consumer preference data and the drug feature data, a plurality of features for a predictive model, training, based on the plurality of features, the predictive model, and outputting the predictive model.

In an embodiment, disclosed are methods comprising receiving, at a computing device, data associated with a prescription drug, determining drug feature data associated with a plurality of drugs, providing, to a predictive model, the data associated with the prescription drug and the drug feature data, and determining, based on the predictive model, at least one candidate drug to purchase and stock.

In an embodiment, disclosed are methods comprising receiving, at a computing device, data associated with a prescription drug, determining drug feature data associated with a plurality of drugs, providing, to a predictive model, the data associated with the prescription drug and the drug feature data, and determining, based on the predictive model, at least one recommendation of at least one substitution drug associated with the prescription drug.

In an embodiment, disclosed are methods comprising receiving, at a computing device, drug preference data associated with at least one consumer, receiving data associated with at least one prescription drug, determining drug feature data associated with a plurality of drugs, providing, to a predictive model, the drug preference data, the data associated with the at least one prescription drug, and the drug feature data, and determining, based on the predictive model, a recommendation of at least one substitution drug.

In an embodiment, disclosed are methods comprising receiving, at a computing device, active ingredient data associated with a prescription drug, determining drug feature data associated with the active ingredient data, providing, to a predictive model, the active ingredient data and the drug feature data, and determining, based on the predictive model, a drug design.

Additional advantages will be set forth in part in the description which follows or may be learned by practice. The advantages will be realized and attained by means of the elements and combinations particularly pointed out in the appended claims.

### BRIEF DESCRIPTION OF THE DRAWINGS

The accompanying drawings, which are incorporated in and constitute a part of the present description serve to explain the principles of the methods and systems described herein:

FIG. 1 shows an example method;

FIG. 2 shows an example machine learning system;

FIG. 3 shows an example machine learning method;

FIG. 4 shows an example product attributes webpage;

FIG. 5 shows an example word count graph associated with a product attribute;

FIG. 6 shows an example computer system for implementing the machine learning system;

FIG. 7 shows an example method;

FIG. 8 shows an example method;

FIG. 9 shows an example method;

FIG. 10 shows an example method; and

FIG. 11 shows an example method.

### DETAILED DESCRIPTION

As used in the specification and the appended claims, the singular forms “a,” “an,” and “the” include plural referents unless the context clearly dictates otherwise. Ranges may be expressed herein as from “about” one particular value, and/or to “about” another particular value. When such a range is expressed, another configuration includes from the one particular value and/or to the other particular value. Similarly, when values are expressed as approximations, by use of the antecedent “about,” it will be understood that the particular value forms another configuration. It will be further understood that the endpoints of each of the ranges are significant both in relation to the other endpoint, and independently of the other endpoint.

“Optional” or “optionally” means that the subsequently described event or circumstance may or may not occur, and that the description includes cases where said event or circumstance occurs and cases where it does not.

Throughout the description and claims of this specification, the word “comprise” and variations of the word, such as “comprising” and “comprises,” means “including but not limited to,” and is not intended to exclude, for example, other components, integers or steps. “Exemplary” means “an example of” and is not intended to convey an indication of a preferred or ideal configuration. “Such as” is not used in a restrictive sense, but for explanatory purposes.

It is understood that when combinations, subsets, interactions, groups, etc. of components are described that, while specific reference of each various individual and collective combinations and permutations of these may not be explicitly described, each is specifically contemplated and described herein. This applies to all parts of this application including, but not limited to, steps in described methods. Thus, if there are a variety of additional steps that may be performed it is understood that each of these additional steps may be performed with any specific configuration or combination of configurations of the described methods.

As will be appreciated by one skilled in the art, hardware, software, or a combination of software and hardware may be implemented. Furthermore, a computer program product on a computer-readable storage medium (e.g., non-transitory) having processor-executable instructions (e.g., computer software) embodied in the storage medium. Any suitable computer-readable storage medium may be utilized including hard disks, CD-ROMs, optical storage devices, magnetic storage devices, memresistors, Non-Volatile Random Access Memory (NVRAM), flash memory, or a combination thereof.

Throughout this application reference is made to block diagrams and flowcharts. It will be understood that each block of the block diagrams and flowcharts, and combinations of blocks in the block diagrams and flowcharts, respectively, may be implemented by processor-executable instructions. These processor-executable instructions may be loaded onto a general purpose computer, special purpose computer, or other programmable data processing apparatus to produce a machine, such that the processor-executable instructions which execute on the computer or other programmable data processing apparatus create a device for implementing the functions specified in the flowchart block or blocks.

These processor-executable instructions may also be stored in a computer-readable memory that may direct a computer or other programmable data processing apparatus to function in a particular manner, such that the processor-executable instructions stored in the computer-readable memory produce an article of manufacture including processor-executable instructions for implementing the function specified in the flowchart block or blocks. The processor-executable instructions may also be loaded onto a computer or other programmable data processing apparatus to cause a series of operational steps to be performed on the computer or other programmable apparatus to produce a computer-implemented process such that the processor-executable instructions that execute on the computer or other programmable apparatus provide steps for implementing the functions specified in the flowchart block or blocks.

Blocks of the block diagrams and flowcharts support combinations of devices for performing the specified functions, combinations of steps for performing the specified functions and program instruction means for performing the specified functions. It will also be understood that each block of the block diagrams and flowcharts, and combinations of blocks in the block diagrams and flowcharts, may be implemented by special purpose hardware-based computer systems that perform the specified functions or steps, or combinations of special purpose hardware and computer instructions.

Methods and systems are described for generating a machine learning classifier for the prediction of expected consumer preference indicative of a prescription drug or at least one substitution drug associated with the prescription drug. The expected consumer preference may be used to predict one or more of when a quantity to restock a desired drug, a recommendation of at least one substitution drug associated with a consumer selection of at least one drug, or a drug design according to the expected consumer preference result. Machine learning (ML) is a subfield of computer science that gives computers the ability to learn without being explicitly programmed. Machine learning platforms include, but are not limited to, naïve Bayes classifiers, support vector machines, decision trees, neural networks, and the like.

In an embodiment, a plurality of consumer preference data associated with at least one prescription drug may be obtained. The plurality of consumer preference data may comprise one or more of an indication of consumer preference for at least one substitution drug of the prescription drug or an indication of consumer preference for no substitution of the prescription drug. The indication of the consumer preference for at least one substitution drug comprises consumer preferences based on one or more of drug color, drug size, drug flavor, drug shape, drug manufacturer, therapeutic class, active ingredients of a drug, or inactive ingredients of a drug. Natural language processing (NLP) may be applied to drug description data (e.g., product attributes) associated with a plurality of drugs. The drug description data may be received from a public data source. The drug feature data may comprise one or more of drug color, drug size, drug flavor, drug shape, drug manufacturer, therapeutic class, active ingredients of a drug, or inactive ingredients of a drug. The machine learning classifier, or predictive model, may be trained using the plurality of consumer preference data and the drug feature data. In an embodiment, the predictive model may comprise a neural network.

The predictive model may output a prediction indicative of an expected consumer preference result. The expected consumer preference result may comprise a prediction indicative of consumer preference for one or more of the prescription drug or at least one substitution associated with the prescription drug. The expected consumer preference result may be further indicative of one or more of one or more of when and a quantity to restock a desired drug, a recommendation of at least one substitution drug associated with a consumer selection of at least one drug, or a drug design according to the expected consumer preference result.

In an embodiment, data associated with at least one prescription drug of a plurality of a plurality of prescription drugs may be obtained. NLP may be applied to drug description data associated with a plurality of drugs. The drug description data may be received from a public data source. The drug feature data may comprise one or more of drug color, drug size, drug flavor, drug shape, drug manufacturer, therapeutic class, active ingredients of a drug, or inactive ingredients of a drug. The data associated with the at least one prescription drug and the drug feature data may be provided to the predictive model. At least one candidate drug may be determined based on the predictive model. The at least one candidate drug may comprise one or more of the at least one prescription drug or at least one substitution drug associated with the at least one prescription drug. The at least one substitution drug may be further associated with the drug feature data.

In an embodiment, data associated with at least one prescription drug of a plurality of prescription drugs may be obtained. NLP may be applied to drug description data associated with a plurality of drugs. The drug description data may be received from a public data source. The drug feature data may comprise one or more of drug color, drug size, drug flavor, drug shape, drug manufacturer, therapeutic class, active ingredients of a drug, or inactive ingredients of a drug. The data associated with the at least one prescription drug and the drug feature data may be provided to the predictive model. At least one recommendation of at least one substitution drug associated with the at least one prescription drug may be determined based on the predictive model.

In an embodiment, preference data associated with at least one consumer may be obtained. The preference data may comprise a preference for one or more drug features. The one or more drug features may include one or more of drug color, drug size, drug flavor, drug shape, drug manufacturer, therapeutic class, active ingredients of a drug, or inactive ingredients of a drug. Further, data associated with at least one prescription drug may be received. NLP may be applied to drug description data associated with a plurality of drugs. The drug description data may be received from a public data source.

The drug feature data may comprise one or more of drug color, drug size, drug flavor, drug shape, drug manufacturer, therapeutic class, active ingredients of a drug, or inactive ingredients of a drug. The preference data, the data associated with the at least one prescription drug, and the drug feature data may be applied to the predictive model, wherein weights associated with features of the predictive model may be adjusted according to the preference data. A recommendation of at least one substitution drug may be determined based on the predictive model.

In an embodiment, active ingredient data associated with at least one prescription drug may be obtained. NLP may be applied to drug description data associated with the active ingredient data. The drug description data associated with the active ingredient data may be received from a public data source. The drug feature data may comprise one or more of drug color, drug size, drug flavor, drug shape, drug manufacturer, therapeutic class, or inactive ingredients of a drug. The active ingredient data and the drug feature data may be provided to the predictive model. A drug design may be determined based on the predictive model. The drug design may comprise an active ingredient associated with the active ingredient data and one or more features associated with the drug feature data.

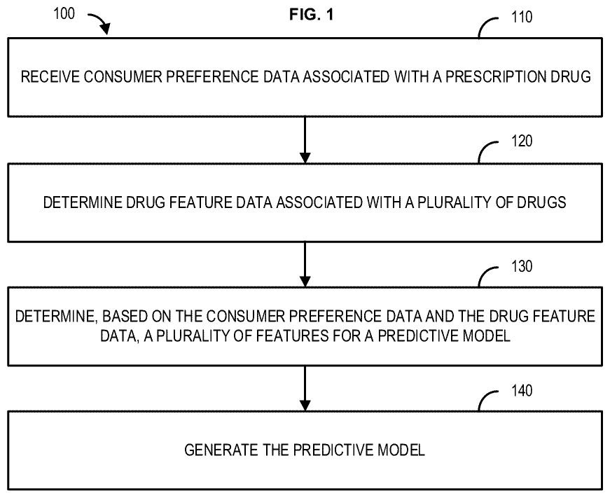

In an embodiment, (shown in FIG. 1) disclosed is a method 100 for generating a predictive model comprising receiving consumer preference data associated with a prescription drug at 110, determining drug feature data associated with a plurality of drugs at 120, generating, based on the consumer preference data and the drug feature data, a plurality of features for a predictive model at 130, and generating the predictive model at 140.

Receiving consumer preference data associated with a prescription drug at 110 may comprise downloading/obtaining/receiving consumer preference data sets, obtained from various sources, including recent publications and/or publically available databases.

The consumer preference data associated with the prescription drug may comprise one or more of an indication of consumer preference for at least one substitution drug of the prescription drug or an indication of consumer preference for no substitution of the prescription drug. The indication of the consumer preference for the at least one substitution drug may comprise consumer preferences for one or more of drug color, drug size, drug flavor, drug shape, drug manufacturer, therapeutic class, active ingredients of a drug, or inactive ingredients of a drug. The methods described herein may utilize consumer preference data associated with one or more prescription drugs to improve the prediction of products to include in a company's portfolio.

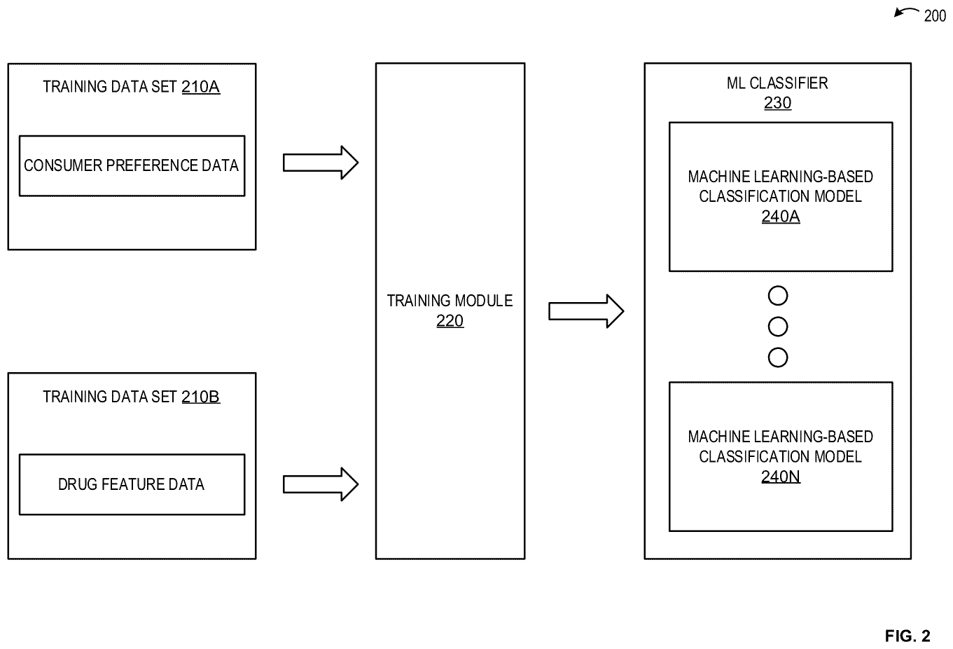

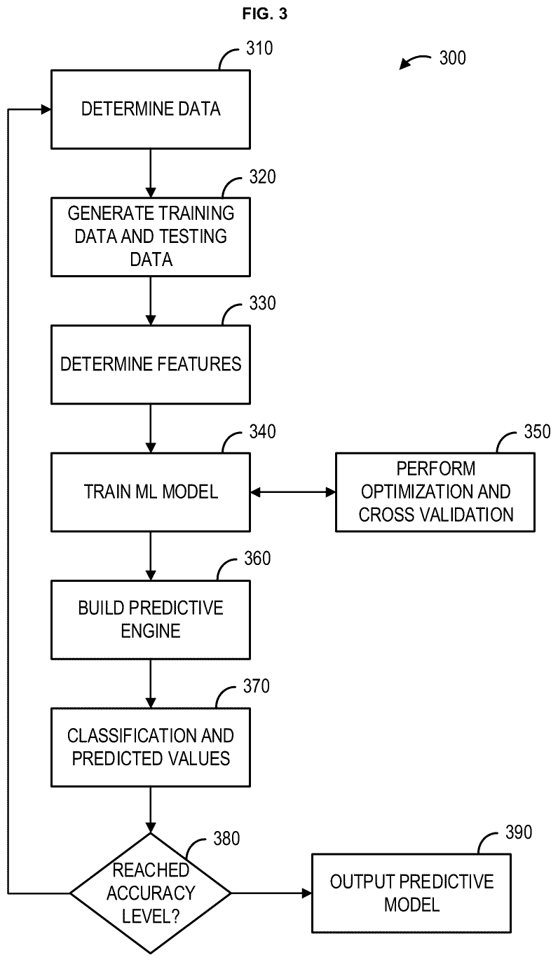

Determining drug feature data associated with a plurality of drugs at 120, generating, based on the consumer preference data and the drug feature data, a plurality of features for a predictive model at 130, and generating the predictive model at 140 are described with regard to FIG. 2 and FIG. 3.

In an embodiment, a predictive model (e.g., a machine learning classifier) may be generated to provide a prediction indicative of an expected consumer preference for one or more of a prescription drug or at least one substitution drug associated with the prescription drug. The predictive model may be trained according to consumer preference data associated with at least one prescription drug (e.g., one or more consumer preference data sets) and drug feature data (e.g., product attributes comprising one or more of drug color, drug size, drug flavor, drug shape, drug manufacturer, therapeutic class, active ingredients of a drug, or inactive ingredients of a drug). In an embodiment, natural language processing may be applied to drug description data associated with a plurality of drugs in order to determine drug feature data associated with the plurality of drugs. In an embodiment, FDA product information, such as product marketing start date and product attribute data for each generic drug (identified by the National Drug Code), may be collected. The product attribute data may include product dose form, pill shape, pill color, product flavor, product size, etc. One or more NLP transformations may be applied to each of the product attributes to clean text data, transform the text data to numeric data for input in the predictive (machine learning) model, and reduce dimensionality (e.g., count vectorizing, term frequency-inverse document frequency, hashing, etc.).

As shown in FIG. 2, a system 200 is described herein that is configured to use machine learning techniques to train, based on an analysis of one or more training data sets 210A-210B by a training module 220, at least one machine learning-based classifier 230 that is configured to classify expected consumer preference results. In an embodiment, the training data set 210A (e.g., the consumer preference data) may comprise the one or more consumer preference data sets. In an embodiment, the consumer preference data sets 210A may comprise one or more of an indication of consumer preference for at least one substitution drug of the prescription drug or an indication of consumer preference for no substitution of the prescription drug. The indication of the consumer preference for at least one substitution drug may comprise one or more of drug color, drug size, drug flavor, drug shape, drug manufacturer, therapeutic class, active ingredients of a drug, or inactive ingredients of a drug. In an embodiment, the training data set 210B may comprise drug feature data associated with a plurality of drugs. In an embodiment, natural language processing (NLP) may be applied to the drug description data (e.g., product attributes) associated with the plurality of drugs to determine the drug feature data. The drug description data may be received from a public data source. FDA product information, such as product marketing start date and product attribute data for each generic drug (identified by the National Drug Code), may be collected. The product attribute data may include product dose form, pill shape, pill color, product flavor, product size, etc. One or more NLP transformations may be applied to each of the product attributes to clean text data, transform the text data to numeric data for input in the predictive (machine learning) model, and reduce dimensionality (e.g., count vectorizing, term frequency-inverse document frequency, hashing, etc.). Product attribute trends may be determined. For instance a time series of product attribute data may be created, wherein an autoregressive model may be used to determine which product attributes are growing in popularity or being phased out.

Historic transactional substitution data, at the National Drug Code (NDC) item level may be aggregated. Utilizing the NDC, the FDA product marketing start data, and the sales transaction date, the FDA product attribute data (e.g., the training data set 210B) may be merged with the consumer preference data (e.g., the training data set 210A) for training the predictive model to identify items patients will prefer based on the product, or drug, attributes.

In an embodiment, the training module 220 may train the machine learning-based classifier 230 by extracting a feature set from the consumer preference data (e.g., one or more consumer preference data sets) in the training data set 210A and the drug feature data (e.g., one or more of drug color, drug size, drug flavor, drug shape, drug manufacturer, therapeutic class, active ingredients of a drug, or inactive ingredients of a drug) in the training data set 210B according to one or more feature selection techniques.

In an embodiment, the training module 220 may extract a feature set from the training data set 210A and/or the training data set 210B in a variety of ways. The training module 220 may perform feature extraction multiple times, each time using a different feature-extraction technique. In an embodiment, the feature sets generated using the different techniques may each be used to generate different machine learning-based classification models 240. In an embodiment, the feature set with the highest quality metrics may be selected for use in training. The training module 220 may use the feature set(s) to build one or more machine learning-based classification models 240A-240N that are configured to indicate an expected consumer preference result. The expected consumer preference result may comprise one or more of the prescription drug or at least one substitution drug associated with the prescription drug. The expected consumer preference result may be used to provide a prediction indicative of one or more of when and a quantity to restock a desired drug, a recommendation of at least one substitution drug associated with a consumer selection of at least one drug, or a drug design according to the expected consumer preference result.

In an embodiment, the training data set 210A may be analyzed to determine one or more groups of consumer preference data that have at least one feature that may be used to predict an expected consumer preference of a prescription drug or a substitution drug associated with the prescription drug. The one or more groups of consumer preference data may be considered as features (or variables) in the machine learning context. The term “feature,” as used herein, may refer to any characteristic of a group, or a series, of consumer preference data of a prescription drug or a substitution drug that may be used to determine whether the group of consumer preference data fall within one or more specific categories. By way of example, the features described herein may comprise one or more group of consumer preference data.

In an embodiment, a feature selection technique may comprise one or more feature selection rules. The one or more feature selection rules may comprise a consumer preference occurrence rule. The consumer preference occurrence rule may comprise determining which consumer preference, or group of consumer preferences, in the training data set 210A occur over a threshold number of times and identifying those consumer preferences that satisfy the threshold as candidate features. For example, any consumer preference, or group of consumer preferences, that appear greater than or equal to 50 times in the training data set 210A may be considered as candidate features. Any consumer preference, or group of consumer preferences, appearing less than 50 times may be excluded from consideration as a feature.

In an embodiment, the one or more feature selection rules may comprise a significance rule. The significance rule may comprise determining, from the consumer preference data in the training data set 210A, one or more of an indication of consumer preference for at least one substitution drug of the prescription drug or an indication of consumer preference for no substitution of the prescription drug. The indication of the consumer preference for at least one substitution drug comprises consumer preferences may be based on one or more of drug color, drug size, drug flavor, drug shape, drug manufacturer, therapeutic class, active ingredients of a drug, or inactive ingredients of a drug.

In an embodiment, a single feature selection rule may be applied to select features or multiple feature selection rules may be applied to select the features. In an embodiment, the feature selection rules may be applied in a cascading fashion, with the feature selection rules being applied in a specific order and applied to the results of the previous rule. For example, the consumer preference occurrence rule may be applied to the training data set 210A to generate a first list of features. The significance rule may be applied to features in the first list of features to determine which features of the first list satisfy the significance rule in the training data set 210A and to generate a final list of candidate features.

The final list of candidate features may be analyzed according to additional feature selection techniques to determine one or more candidate feature signatures (e.g., groups, or series, of consumer preferences that may be used to predict an expected consumer preference result). Any suitable computational technique may be used to identify the candidate feature signatures using any feature selection technique such as filter, wrapper, and/or embedded methods. In an embodiment, one or more candidate feature signatures may be selected according to a filter method. Filter methods include, for example, Pearson's correlation, linear discriminant analysis, analysis of variance (ANOVA), chi-square, combinations thereof, and the like. The selection of features according to filter methods are independent of any machine learning algorithms. Instead, features may be selected on the basis of scores in various statistical tests for their correlation with the outcome variable (e.g., an expected consumer preference result).

In an embodiment, one or more candidate feature signatures may be selected according to a wrapper method. A wrapper method may be configured to use a subset of features and train a machine learning model using the subset of features. Based on the inferences that are drawn from a previous model, features may be added and/or deleted from the subset. Wrapper methods include, for example, forward feature selection, backward feature elimination, recursive feature elimination, combinations thereof, and the like. In an embodiment, forward feature selection may be used to identify one or more candidate feature signatures. Forward feature selection is an iterative method that begins with no feature in the machine learning model. In each iteration, the feature which best improves the model is added until an addition of a new variable does not improve the performance of the machine learning model. In an embodiment, backward elimination may be used to identify one or more candidate feature signatures. Backward elimination is an iterative method that begins with all features in the machine learning model. In each iteration, the least significant feature is removed until no improvement is observed on removal of features. In an embodiment, recursive feature elimination may be used to identify one or more candidate feature signatures. Recursive feature elimination is a greedy optimization algorithm which aims to find the best performing feature subset. Recursive feature elimination repeatedly creates models and keeps aside the best or the worst performing feature at each iteration. Recursive feature elimination constructs the next model with the features remaining until all the features are exhausted.

Recursive feature elimination then ranks the features based on the order of their elimination.

In an embodiment, one or more candidate feature signatures may be selected according to an embedded method. Embedded methods combine the qualities of filter and wrapper methods. Embedded methods include, for example, Least Absolute Shrinkage and Selection Operator (LASSO) and ridge regression which implement penalization functions to reduce overfitting. For example, LASSO regression performs L1 regularization which adds a penalty equivalent to absolute value of the magnitude of coefficients and ridge regression performs L2 regularization which adds a penalty equivalent to square of the magnitude of coefficients.

After the training module 220 has generated a feature set(s), the training module 220 may generate a machine learning-based classification model 240 based on the feature set(s). The machine learning-based classification model 240, may refer to a complex mathematical model for data classification that is generated using machine-learning techniques. In one example, this machine learning-based classifier may include a map of support vectors that represent boundary features. By way of example, boundary features may be selected from, and/or represent the highest-ranked features in, a feature set.

In an embodiment, the training module 220 may use the feature sets extracted from the training data set 210A and/or the training data set 210B to build a machine learning-based classification model 240A-240N for each classification category (e.g., each expected consumer preference result). In some examples, the machine learning-based classification models 240A-240N may be combined into a single machine learning-based classification model 240. Similarly, the machine learning-based classifier 230 may represent a single classifier containing a single or a plurality of machine learning-based classification models 240 and/or multiple classifiers containing a single or a plurality of machine learning-based classification models 240.

The extracted features (e.g., consumer preference data and/or drug feature data) may be combined in a classification model trained using a machine learning approach such as discriminant analysis; decision tree; a nearest neighbor (NN) algorithm (e.g., k-NN models, replicator NN models, etc.); statistical algorithm (e.g., Bayesian networks, etc.); clustering algorithm (e.g., k-means, mean-shift, etc.); neural networks (e.g., reservoir networks, artificial neural networks, etc.); support vector machines (SVMs); logistic regression algorithms; linear regression algorithms; Markov models or chains; principal component analysis (PCA) (e.g., for linear models); multi-layer perceptron (MLP) ANNs (e.g., for non-linear models); replicating reservoir networks (e.g., for non-linear models, typically for time series); random forest classification; a combination thereof and/or the like. The resulting machine learning-based classifier 230 may comprise a decision rule or a mapping that uses the expression levels of the features in the candidate feature signature to predict an expected consumer preference result.

The candidate feature signature and the machine learning-based classifier 230 may be used to provide a prediction indicative of an expected consumer preference result in the testing data set. In one example, the result for each test includes a confidence level that corresponds to a likelihood or a probability that the corresponding test predicted an expected consumer preference result. The confidence level may be a value between zero and one that represents a likelihood that the corresponding test is associated with an expected consumer preference result. In one example, when there are two or more statuses (e.g., two or more expected consumer preference results), the confidence level may correspond to a value p, which refers to a likelihood that a particular test is associated with a first status. In this case, the value I-p may refer to a likelihood that the particular test is associated with a second status. In general, multiple confidence levels may be provided for each test and for each candidate feature signature when there are more than two statuses. A top performing candidate feature signature may be determined by comparing the result obtained for each test with known expected consumer preference results for each test. In general, the top performing candidate feature signature will have results that closely match the known consumer preferences.

The top performing candidate feature signature may be used to predict the expected consumer preference result. For example, consumer preference data and drug feature data may be determined/received. The consumer preference data and the drug feature data may be provided to the machine learning-based classifier 230 which may, based on the top performing candidate feature signature, predict/determine an expected consumer preference result. The expected consumer preference result may comprise one or more of a preference for the prescription drug or at least one substitution drug associated with the prescription drug. The expected consumer preference result may be used for a prediction indicative of one or more of when and a quantity to restock a desired drug, a recommendation of at least one substitution drug associated with a consumer selection of at least one drug, or a drug design according to the expected consumer preference result.

FIG. 3 is a flowchart illustrating an example training method 300 for generating the machine learning-based classifier 230 using the training module 220. The training module 220 can implement using supervised, unsupervised, and/or semi-supervised (e.g., reinforcement based) machine learning-based classification models 240. The method 300 illustrated in FIG. 3 is an example of a supervised learning method; variations of this example of training method are discussed below, however, other training methods can be analogously implemented to train unsupervised and/or semi-supervised machine learning models.

The training method 300 may determine (e.g., access, receive, retrieve, etc.) consumer preference data comprising one or more of an indication of consumer preference for at least one substitution drug of the prescription drug or an indication of consumer preference for no substitution of the prescription drug at 310. The indication of the consumer preference for at least one substitution drug comprises consumer preferences based on one or more of drug color, drug size, drug flavor, drug shape, drug manufacturer, therapeutic class, active ingredients of a drug, or inactive ingredients of a drug. In an embodiment, the consumer preference data may include studies pertaining to historical consumer transactional data. In an embodiment, each dataset may include a labeled list of predetermined features. In addition to the consumer preference data, drug feature data may be determined at 310. In an embodiment, natural language processing (NLP) may be applied to drug description data (e.g., product attributes) associated with the plurality of drugs to determine the drug feature data. The drug description data may be received from a public data source. FDA product information, such as product marketing start date and product attribute data for each generic drug (identified by the National Drug Code), may be collected. The product attribute data may include product dose form, pill shape, pill color, product flavor, product size, etc. One or more NLP transformations may be applied to each of the product attributes to clean the text data, transform the text data to numeric data for input in the predictive model, and reduce dimensionality (e.g., count vectorizing, term frequency-inverse document frequency, hashing, etc.). Product attribute trends may be determined. For instance a time series of product attribute data may be created, wherein an autoregressive model may be used to determine which product attributes are growing in popularity or being phased out. Historic transactional substitution data, at the National Drug Code (NDC) item level may be aggregated. Utilizing the NDC, the FDA product marketing start data, and the sales transaction date, the FDA product attribute data may be merged with the consumer preference data for training the predictive model to identify items consumers will prefer based on the product, or drug, attributes.

The training method 300 may generate, at 320, a training data set and a testing data set. The training data set and the testing data set may be generated by randomly assigning labeled feature data of individual features from the consumer preference data and the drug feature data to either the training data set or the testing data set. In some implementations, the assignment of the labeled feature data of individual features may not be completely random. In an embodiment, only the labeled feature data for a specific study may be used to generate the training data set and the testing data set. In an embodiment, a majority of the labeled feature data for the specific study may be used to generate the training data set. For example, 75% of the labeled feature data for the specific study may be used to generate the training data set and 25% may be used to generate the testing data set. In another embodiment, only the labeled feature data for the specific study may be used to generate the training data set and the testing data set.

The training method 300 may determine (e.g., extract, select, etc.), at 330, one or more features that can be used by, for example, a classifier to differentiate among different classifications (e.g., different expected consumer preference results). The one or more features may comprise a group of consumer preference data and/or a group of drug feature data. In an embodiment, the training method 300 may determine a set of features from the consumer preference data and the drug feature data. In another embodiment, a set of features may be determined from consumer preference data and drug feature data from a study different than the study associated with the labeled feature data of the training data set and the testing data set. In other words, the consumer preference data and the drug feature data from the different study (e.g., curated parameter data sets) may be used for feature determination, rather than for training a machine learning model. In an embodiment, the training data set may be used in conjunction with the consumer preference data and the drug feature data from the different study to determine the one or more features. The consumer preference data and the drug feature data from the different study may be used to determine an initial set of features, which may be further reduced using the training data set.

The training method 300 may train one or more machine learning models using the one or more features at 340. In one embodiment, the machine learning models may be trained using supervised learning. In another embodiment, other machine learning techniques may be employed, including unsupervised learning and semi-supervised. The machine learning models trained at 340 may be selected based on different criteria depending on the problem to be solved and/or data available in the training data set. For example, machine learning classifiers can suffer from different degrees of bias. Accordingly, more than one machine learning model can be trained at 340, optimized, improved, and cross-validated at 350.

The training method 300 may select one or more machine learning models to build a predictive model at 360 (e.g., a machine learning classifier). The predictive model may be evaluated using the testing data set. The predictive model may analyze the testing data set and generate classification values and/or predicted values at 370. Classification and/or prediction values may be evaluated at 380 to determine whether such values have achieved a desired accuracy level. Performance of the predictive model may be evaluated based on future, or new, drug transactional data to measure the success of the expected consumer preference result. For example, future transactional data may indicate a consumer preference for a prescription drug that was predicted by the predictive model. In another example, future transaction data may indicate a consumer preference for a substitution drug when the predictive model indicated a preference for the prescription drug.

When a desired accuracy level is reached, the training phase ends and the predictive model may be output at 390; when the desired accuracy level is not reached, however, then a subsequent iteration of the training method 300 may be performed starting at 310 with variations such as, for example, considering a larger collection of consumer preference data.

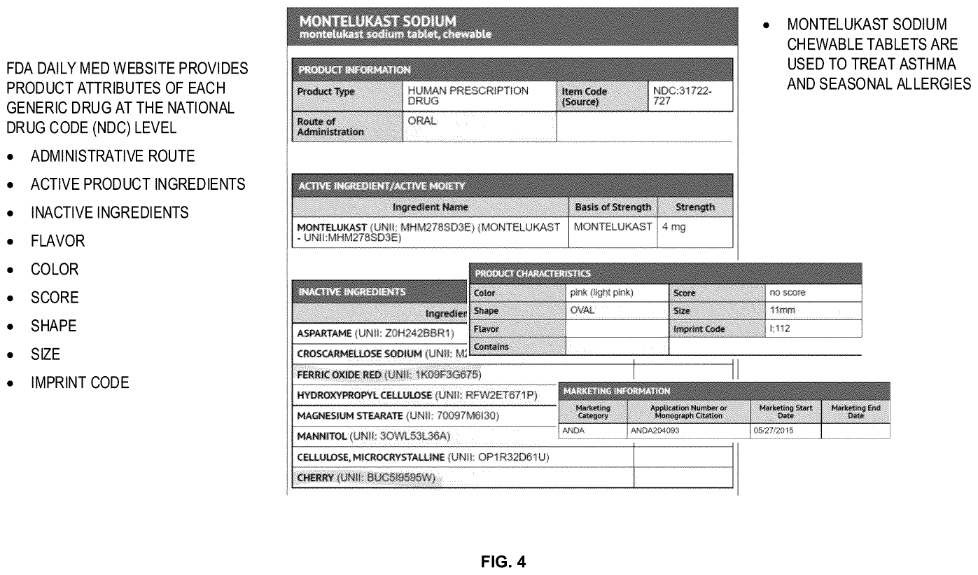

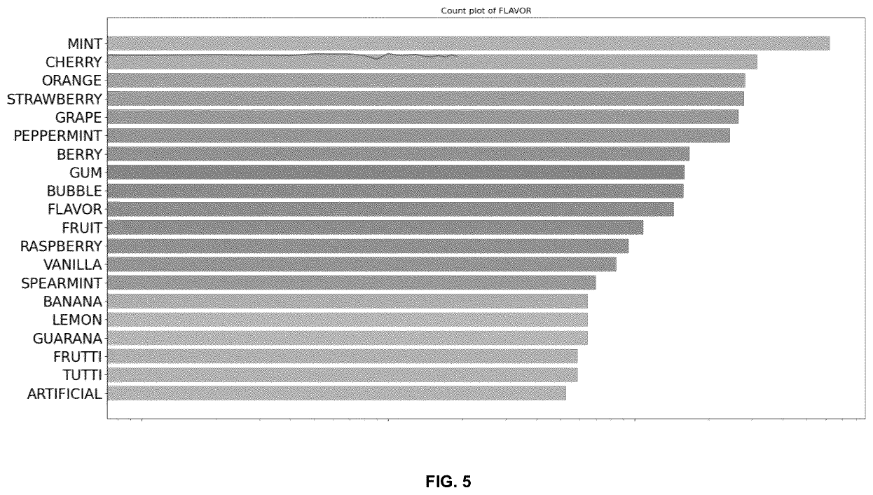

FIG. 4 shows an example of product attributes associated with a particular drug, Montelukast Sodium chewable tablets. The FDA daily medical website provides product attributes for each generic drug at the National Drug Code (NDC) level. For instance, the website indicates the administrative route, active product ingredients, inactive ingredients, flavor, color, score, shape, size, and imprint code. One or more NLP transformations may be applied to each of the product attributes found on the website in order to clean text data, transform the text data to numeric data (e.g., drug feature data) for input in the predictive (machine learning) model, and reduce dimensionality (e.g., count vectorizing, term frequency-inverse document frequency, hashing, etc.). For instance, by applying NLP to the product attributes word counts associated with the product attributes. FIG. 5 shows an example of a word count plot related to the product flavor attribute. The drug feature data may be combined, or merged, with the consumer preference data, wherein this data may be used to train a predictive model.

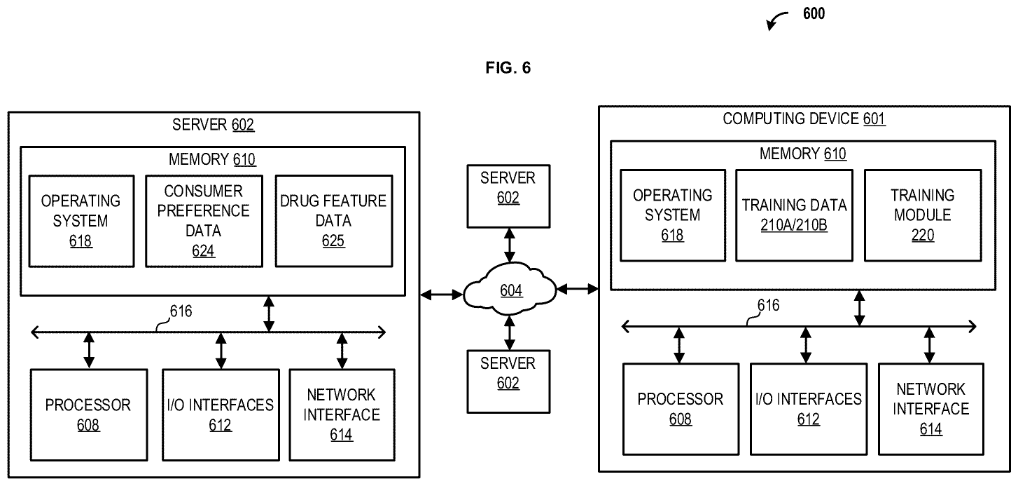

FIG. 6 is a block diagram depicting an environment 600 comprising non-limiting examples of a computing device 601 and a server 602 connected through a network 604. In an aspect, some or all steps of any described method may be performed on a computing device as described herein. The computing device 601 can comprise one or multiple computers configured to store one or more of the training module 220, training data 210A/210B (e.g., consumer preference data and/or drug feature data), and the like. The server 602 can comprise one or multiple computers configured to store the consumer preference data 624 and/or the drug feature data 625. Multiple servers 602 can communicate with the computing device 601 via the through the network 604.

The computing device 601 and the server 602 can be a digital computer that, in terms of hardware architecture, generally includes a processor 608, memory system 610, input/output (I/O) interfaces 612, and network interfaces 614. These components (608, 610, 612, and 614) are communicatively coupled via a local interface 616. The local interface 616 can be, for example, but not limited to, one or more buses or other wired or wireless connections, as is known in the art. The local interface 616 can have additional elements, which are omitted for simplicity, such as controllers, buffers (caches), drivers, repeaters, and receivers, to enable communications. Further, the local interface may include address, control, and/or data connections to enable appropriate communications among the aforementioned components.

The processor 608 can be a hardware device for executing software, particularly that stored in memory system 610. The processor 608 can be any custom made or commercially available processor, a central processing unit (CPU), an auxiliary processor among several processors associated with the computing device 601 and the server 602, a semiconductor-based microprocessor (in the form of a microchip or chip set), or generally any device for executing software instructions. When the computing device 601 and/or the server 602 is in operation, the processor 608 can be configured to execute software stored within the memory system 610, to communicate data to and from the memory system 610, and to generally control operations of the computing device 601 and the server 602 pursuant to the software.

The I/O interfaces 612 can be used to receive user input from, and/or for providing system output to, one or more devices or components. User input can be provided via, for example, a keyboard and/or a mouse. System output can be provided via a display device and a printer (not shown). I/O interfaces 612 can include, for example, a serial port, a parallel port, a Small Computer System Interface (SCSI), an infrared (IR) interface, a radio frequency (RF) interface, and/or a universal serial bus (USB) interface.

The network interface 614 can be used to transmit and receive from the computing device 601 and/or the server 602 on the network 604. The network interface 614 may include, for example, a 10 BaseT Ethernet Adaptor, a 100 BaseT Ethernet Adaptor, a LAN PHY Ethernet Adaptor, a Token Ring Adaptor, a wireless network adapter (e.g., WiFi, cellular, satellite), or any other suitable network interface device. The network interface 614 may include address, control, and/or data connections to enable appropriate communications on the network 604.

The memory system 610 can include any one or combination of volatile memory elements (e.g., random access memory (RAM, such as DRAM, SRAM, SDRAM, etc.)) and nonvolatile memory elements (e.g., ROM, hard drive, tape, CDROM, DVDROM, etc.). Moreover, the memory system 610 may incorporate electronic, magnetic, optical, and/or other types of storage media. Note that the memory system 610 can have a distributed architecture, where various components are situated remote from one another, but can be accessed by the processor 608.

The software in memory system 610 may include one or more software programs, each of which comprises an ordered listing of executable instructions for implementing logical functions. In the example of FIG. 6, the software in the memory system 610 of the computing device 601 can comprise the training module 220 (or subcomponents thereof), the training data 210A/210B, and a suitable operating system (O/S) 618. In the example of FIG. 6, the software in the memory system 610 of the server 602 can comprise, the consumer preference data 624, the drug feature data 625, and a suitable operating system (O/S) 618. The operating system 618 essentially controls the execution of other computer programs and provides scheduling, input-output control, file and data management, memory management, and communication control and related services.

For purposes of illustration, application programs and other executable program components such as the operating system 618 are illustrated herein as discrete blocks, although it is recognized that such programs and components can reside at various times in different storage components of the computing device 601 and/or the server 602. An implementation of the training module 220 can be stored on or transmitted across some form of computer readable media. Any of the disclosed methods can be performed by computer readable instructions embodied on computer readable media. Computer readable media can be any available media that can be accessed by a computer. By way of example and not meant to be limiting, computer readable media can comprise “computer storage media” and “communications media.” “Computer storage media” can comprise volatile and non-volatile, removable and non-removable media implemented in any methods or technology for storage of information such as computer readable instructions, data structures, program modules, or other data. Exemplary computer storage media can comprise RAM, ROM, EEPROM, flash memory or other memory technology, CD-ROM, digital versatile disks (DVD) or other optical storage, magnetic cassettes, magnetic tape, magnetic disk storage or other magnetic storage devices, or any other medium which can be used to store the desired information and which can be accessed by a computer.

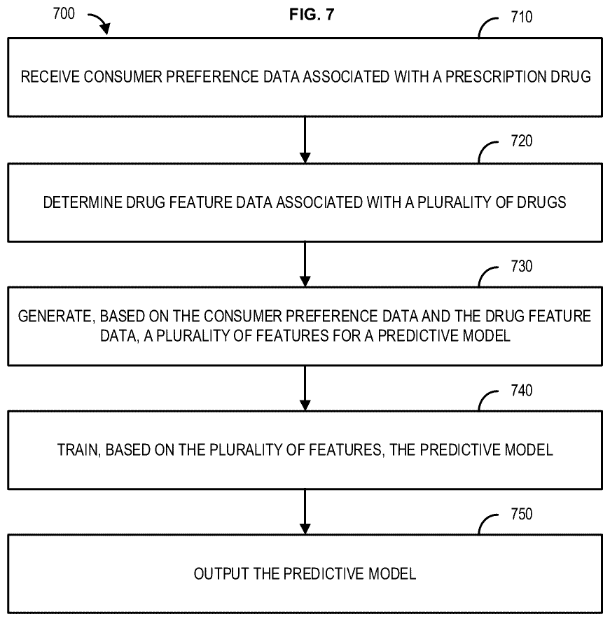

In an embodiment, the training module 220 may be configured to perform a method 700, shown in FIG. 7. The method 700 may be performed in whole, or in part, by a single computing device, a plurality of electronic devices, and the like. The method 700 may comprise receiving consumer preference data associated with a prescription drug at 710. The consumer preference data may comprise one or more of an indication of consumer preference for at least one substitution drug of the prescription drug or an indication of consumer preference for no substitution of the prescription drug. The indication of the consumer preference for at least one substitution drug may comprise consumer preferences based on one or more of drug color, drug size, drug flavor, drug shape, drug manufacturer, therapeutic class, active ingredients of a drug, or inactive ingredients of a drug.

The method 700 may comprise determining drug feature data associated with a plurality of drugs at 720. The drug feature data may be determined based on applying natural language processing (NLP) to drug description data (e.g., product attributes). The drug description data may be received from a public data source, such as the FDA daily medical website. FDA product information, such as product marketing start date and product attribute data for each generic drug (identified by the National Drug Code), may be collected. The product attribute data may include product dose form, pill shape, pill color, product flavor, product size, etc. One or more NLP transformations may be applied to each of the product attributes to clean the text data, transform the text data to numeric data for input in the predictive (machine learning) model, and reduce dimensionality (e.g., count vectorizing, term frequency-inverse document frequency, hashing, etc.). The drug feature data may be combined, or merged, with the consumer preference data for training the predictive model to identify products patients will prefer based on the product, or drug, attributes.

The method 700 may comprise generating, based on the consumer preference data and the drug feature data, a plurality of features for a predictive model at 730. The method 700 may comprise training, based on the plurality of features, the predictive model at 740. In an embodiment, the predictive model may comprise a neural network.

The method 700 may comprise outputting the predictive model at 750. The predictive may be configured to output a prediction of an expected consumer preference result for one or more of the prescription drug or at least one substitution drug associated with the prescription drug. The expected consumer preference result may be further indicative of one or more of when and a quantity to stock, or restock, a desired drug, a recommendation of at least one substitution drug associated with a consumer selection of at least one drug, or a drug design according to the expected consumer preference result.

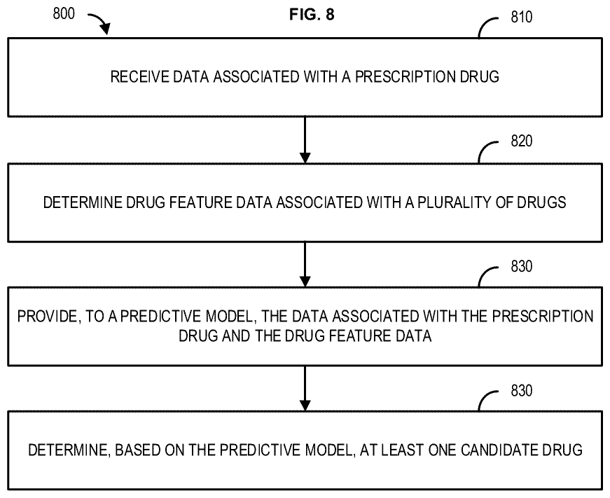

In an embodiment, the training module 220 may be configured to perform a method 800, shown in FIG. 8. The method 800 may be performed in whole, or in part, by a single computing device, a plurality of electronic devices, and the like. The method 800 may comprise receiving data associated a prescription drug at 810.

The method 800 may comprise determining drug feature data associated with a plurality of drugs at 820. The drug feature data may be determined based on applying NLP to drug description data (e.g., product attributes) associated with the plurality of drugs. The plurality of drugs may include generic drugs that may contain the same active ingredients as the prescription drug, wherein the generic drugs may be used as a substitution for the prescription drug based on the shared active ingredients. The drug description data may be received from a public data source, such as the FDA daily medical website. FDA product information, such as product marketing start date and product attribute data for each generic drug (identified by the National Drug Code), may be collected. The product attribute data may include product dose form, pill shape, pill color, product flavor, product size, etc. One or more NLP transformations may be applied to each of the product attributes to clean text data, transform the text data to numeric data for input in the predictive (machine learning) model, and reduce dimensionality (e.g., count vectorizing, term frequency-inverse document frequency, hashing, etc.). The drug feature data may be combined, or merged, with the consumer preference data and provided to the predictive model.

The method 800 may comprise providing, to the predictive model, the data associated with the prescription drug and the drug feature data at 830. The method 800 may further comprise determining, based on the predictive model, at least one candidate drug at 840. The candidate drug may comprise one or more of the prescription drug or at least one substitution drug associated with the prescription drug. The at least one substitution drug may be associated with the drug feature data. For example, the substitution drug may comprise the same active ingredients as the prescription drug. However, the substitution drug may have been predicted based on a consumer preference of one or more of drug color, drug size, drug flavor, drug shape, drug manufacturer, therapeutic class, active ingredients of a drug, or inactive ingredients of a drug. For example, consumer preference may indicate a preference for a substitution drug of a specified prescription drug. In addition, the consumer preference may indicate a preference for a grape-flavored drug from a specified manufacturer. Thus, a grape-flavored substitution drug made by the specified manufacturer may be predicted for inclusion in a company's portfolio based on the specified prescription drug.

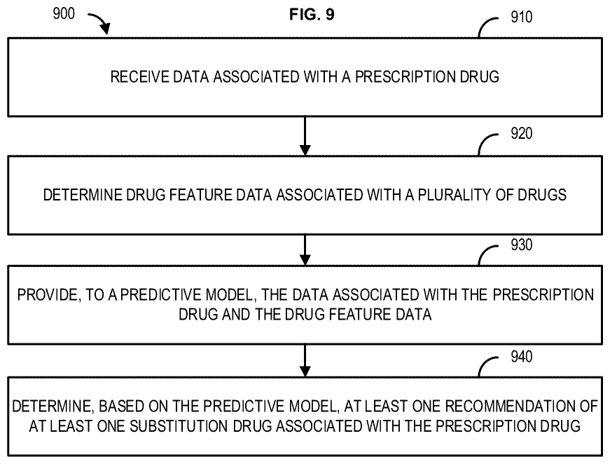

In an embodiment, the training module 220 may be configured to perform a method 900, shown in FIG. 9. The method 900 may be performed in whole, or in part, by a single computing device, a plurality of electronic devices, and the like. The method 900 may comprise receiving data associated a prescription drug at 910.

The method 900 may comprise determining drug feature data associated with a plurality of drugs at 920. The drug feature data may be determined based on applying NLP to drug description data (e.g., product attributes) associated with the plurality of drugs. The plurality of drugs may include generic drugs that may contain the same active ingredients as the prescription drug, wherein the generic drugs may be used as a substitution for the prescription drug based on the shared active ingredients. The drug description data may be received from a public data source, such as the FDA daily medical website. FDA product information, such as product marketing start date and product attribute data for each generic drug (identified by the National Drug Code), may be collected. The product attribute data may include product dose form, pill shape, pill color, product flavor, product size, etc. One or more NLP transformations may be applied to each of the product attributes to clean text data, transform the text data to numeric data for input in the predictive (machine learning) model, and reduce dimensionality (e.g., count vectorizing, term frequency-inverse document frequency, hashing, etc.). The drug feature data may be combined, or merged, with the consumer preference data and provided to the predictive model.

The method 900 may comprise providing, to the predictive model, the data associated with the prescription drug and the drug feature data at 930. The method 900 may further comprise determining, based on the predictive model, at least one recommendation of at least one substitution drug associated with the prescription drug at 940. The recommendation may comprise one or more of the prescription drug or at least one substitution drug associated with the prescription drug. The at least one substitution drug may be associated with the drug feature data. For example, the substitution drug may comprise the same active ingredients as the prescription drug. However, the substitution drug may have been predicted based on a consumer preference of one or more of drug color, drug size, drug flavor, drug shape, drug manufacturer, therapeutic class, active ingredients of a drug, or inactive ingredients of a drug. For example, consumer preference may indicate a preference for substitution drugs of the prescription drug. In addition, the consumer preference may indicate a preference for an oval-shaped and red-colored drug. Thus, an oval-shaped and red-colored substitution drug may be recommended based on a consumer's selection of a prescription drug.

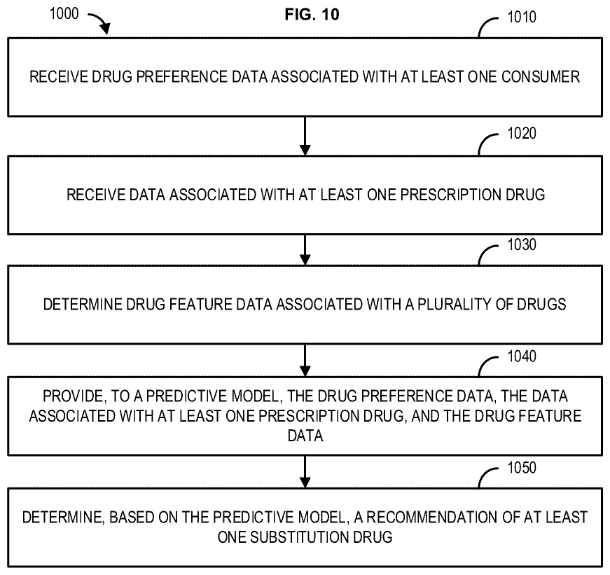

In an embodiment, the training module 220 may be configured to perform a method 1000, shown in FIG. 10. The method 1000 may be performed in whole, or in part, by a single computing device, a plurality of electronic devices, and the like. The method 1000 may comprise receiving drug preference data from at least one consumer at 1010. The drug preference data may be associated with a drug preference for one or more drug features. The one or more drug features may comprise one or more of drug color, drug size, drug flavor, drug shape, drug manufacturer, therapeutic class, active ingredients of a drug, or inactive ingredients of a drug.

The method 1000 may comprise receiving data associated with at least one prescription drug at 1020. The data associated with the at least one prescription drug may comprise active ingredient data. In an embodiment, the data associated with the at least one prescription drug may include drug features comprising one or more drug color, drug size, drug flavor, drug shape, drug manufacturer, therapeutic class, active ingredients of a drug, or inactive ingredients of a drug.

The method 1000 may comprise determining drug feature data associated with a plurality of drugs at 1030. The drug feature data may be determined based on applying NLP to drug description data (e.g., product attributes) associated with the plurality of drugs. The plurality of drugs may include generic drugs that may contain the same active ingredients as a prescription drug, wherein the generic drugs may be used as a substitution for the prescription drug based on the shared active ingredients. The drug description data may be received from a public data source, such as the FDA daily medical website. FDA product information, such as product marketing start date and product attribute data for each generic drug (identified by the National Drug Code), may be collected. The product attribute data may include product dose form, pill shape, pill color, product flavor, product size, etc. One or more NLP transformations may be applied to each of the product attributes to clean text data, transform the text data to numeric data for input in the predictive (machine learning) model, and reduce dimensionality (e.g., count vectorizing, term frequency-inverse document frequency, hashing, etc.). The drug feature data may be combined, or merged, with the consumer preference data and the data associated with the prescription drug and provided to the predictive model.

The method 1000 may comprise providing, to the predictive model, the drug preference data, the data associated with the at least one prescription drug, and the drug feature data at 1040. The method 1000 may further comprise determining, based on the predictive model, at least one recommendation of at least one substitution drug associated with the prescription drug at 1050. Weights associated with features of the predictive model may be adjusted according to the drug preference data in order to personalize the recommendation according to the specific consumer's preferences. The recommendation may comprise one or more of the prescription drug or at least one substitution drug associated with the prescription drug. The at least one substitution drug may be associated with the drug feature data. For example, the substitution drug may comprise the same active ingredients as the prescription drug. However, the substitution drug may have been predicted based on a consumer preference of one or more of drug color, drug size, drug flavor, drug shape, drug manufacturer, therapeutic class, active ingredients of a drug, or inactive ingredients of a drug. For example, the consumer's drug preference data may indicate a preference for substitution drugs of a prescription drug. In addition, the consumer's drug preference data may indicate a preference for a square-shaped and purple-colored drug. Thus, a square-shaped and purple-colored substitution drug may be recommended based on the consumer's selection of a prescription drug and based on the consumer's drug preferences.

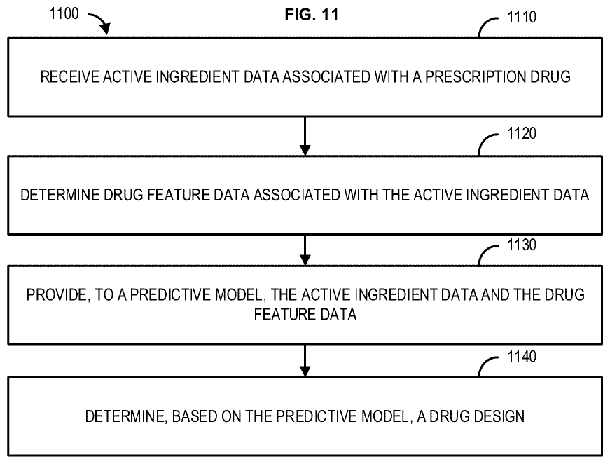

In an embodiment, the training module 220 may be configured to perform a method 1100, shown in FIG. 11. The method 1100 may be performed in whole, or in part, by a single computing device, a plurality of electronic devices, and the like. The method 1100 may comprise receiving active ingredient data associated with a prescription drug at 1110.

The method 1100 may comprise determining drug feature data associated with the active ingredient data at 1120. The drug feature data may be determined based on applying NLP to drug description data (e.g., product attributes) associated with a plurality of drugs associated with the active ingredient data. The plurality of drugs may include generic drugs that may contain the same active ingredients as the prescription drug, wherein the generic drugs may be used as a substitution for the prescription drug based on the shared active ingredients. The drug description data may be received from a public data source, such as the FDA daily medical website. FDA product information, such as product marketing start date and product attribute data for each generic drug (identified by the National Drug Code), may be collected. The product attribute data may include product dose form, pill shape, pill color, product flavor, product size, etc. One or more NLP transformations may be applied to each of the product attributes to clean text data, transform the text data to numeric data for input in the predictive (machine learning) model, and reduce dimensionality (e.g., count vectorizing, term frequency-inverse document frequency, hashing, etc.). The drug feature data may be combined, or merged, with the active ingredient data associated with the prescription drug and provided to the predictive model.

The method 1100 may comprise providing, to the predictive model, the active ingredient data and the drug feature data at 1130. The method 1100 may further comprise determining, based on the predictive model, a drug design at 1140. The drug design may comprise the active ingredient(s) associated with the active ingredient data and one or more features associated with the drug feature data. For example, the drug features may comprise one or more of drug color, drug size, drug flavor, drug shape, drug manufacturer, therapeutic class, active ingredients of a drug, or inactive ingredients of a drug. In an example, consumer drug preference data may indicate a preference for a small circle-shaped drug. Thus, a drug may be designed to include the active ingredient and designed into a small circle-shaped design based on the consumer preference data

Training the predictive model may comprise receiving consumer preference data associated with a prescription drug, wherein the consumer preference data comprises one or more of an indication of consumer preference for at least one substitution drug of the prescription drug or an indication of consumer preference for no substitution of the prescription drug, determining, based on an application of natural language processing (NLP) to drug description data associated with a plurality of drugs, drug feature data associated with the plurality of drugs, generating, based on the consumer preference data and the drug feature data, a plurality of features for a predictive model, training, based on the plurality of features, the predictive model, and outputting the predictive model.

The drug feature data may comprise one or more of drug color, drug size, drug flavor, drug shape, drug manufacturer, therapeutic class, active ingredients of a drug, or inactive ingredients of a drug. The predictive model may be configured to output a prediction indicative of an expected consumer preference result for one or more of the prescription drug or a substitution drug associated with the prescription drug. In an embodiment, the predictive model is configured to output a prediction indicative of one or more of when and a quantity to restock a desired drug, a recommendation of one or more substitution drugs associated with a consumer selection of a drug, or a drug design associated with the prescription drug or the one or more substitution drugs.

While the methods and systems have been described in connection with preferred embodiments and specific examples, it is not intended that the scope be limited to the particular embodiments set forth, as the embodiments herein are intended in all respects to be illustrative rather than restrictive.

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is in no way intended that an order be inferred, in any respect. This holds for any possible non-express basis for interpretation, including: matters of logic with respect to arrangement of steps or operational flow; plain meaning derived from grammatical organization or punctuation; the number or type of embodiments described in the specification.

It will be apparent to those skilled in the art that various modifications and variations can be made without departing from the scope or spirit. Other embodiments will be apparent to those skilled in the art from consideration of the specification and practice disclosed herein. It is intended that the specification and examples be considered as exemplary only, with a true scope and spirit being indicated by the following claims.

1. A method comprising:
receiving, by a computing device, consumer preference data associated with a prescription drug;
determining drug feature data associated with a plurality of drugs;
generating, based on the consumer preference data and the drug feature data, a plurality of features for a predictive model;
training, based on the plurality of features, the predictive model; and
outputting the predictive model.

2. The method of claim 1, wherein the consumer preference data comprises one or more of an indication of consumer preference for at least one substitution drug of the prescription drug or an indication of consumer preference for no substitution of the prescription drug.

3. The method of claim 2, wherein the indication of the consumer preference for at least one substitution drug comprises consumer preferences based on one or more of drug color, drug size, drug flavor, drug shape, drug manufacturer, therapeutic class, active ingredients of a drug, or inactive ingredients of a drug.

4. The method of claim 1, determining the drug feature data associated with the plurality of drugs comprises determining, based on an application of natural language processing (NLP) to drug description data associated with the plurality of drugs, the drug feature data associated with the plurality of drugs.

5. The method of claim 4, wherein the drug description data associated with the plurality of drugs is received from a public data source.

6. The method of claim 1, wherein the drug feature data comprise one or more of drug color, drug size, drug flavor, drug shape, drug manufacturer, therapeutic class, active ingredients of a drug, or inactive ingredients of a drug.

7. The method of claim 1, wherein the predictive model comprises a neural network.

8. The method of claim 1, wherein the predictive model is configured to output a prediction indicative of an expected consumer preference result for one or more of the prescription drug or a substitution drug associated with the prescription drug.

9. The method of claim 1, wherein the predictive model is configured to output a prediction indicative of one or more of when and a quantity to restock a desired drug, a recommendation of one or more substitution drugs associated with a consumer selection of a drug, or a drug design associated with the prescription drug or the one or more substitution drugs.

10. A method comprising:
receiving, at a computing device, data associated with a prescription drug;
determining drug feature data associated with a plurality of drugs;
providing, to a predictive model, the data associated with the prescription drug and the drug feature data; and
determining, based on the predictive model, at least one candidate drug.

11. The method of claim 10, wherein determining the drug feature data associated with the plurality of drugs comprises determining, based on an application of natural language processing (NLP) to drug description data associated with the plurality of drugs.

12. The method of claim 11, wherein the drug description data associated with the plurality of drugs is received from a public data source.

13. The method of claim 10, wherein the drug feature data comprise one or more of drug color, drug size, drug flavor, drug shape, drug manufacturer, therapeutic class, active ingredients of a drug, or inactive ingredients of a drug.

14. The method of claim 10, wherein the least one candidate drug comprises one or more of the prescription drug or at least one substitution drug associated with the prescription drug.

15. The method of claim 14, wherein the at least one substitution drug is associated with the drug feature data.

16. A method comprising:
receiving, at a computing device, data associated with a prescription drug;
determining drug feature data associated with a plurality of drugs;
providing, to a predictive model, the data associated with the prescription drug and the drug feature data; and
determining, based on the predictive model, at least one recommendation of at least one substitution drug associated with the prescription drug.

17. The method of claim 16, wherein determining the drug feature data associated with the plurality of drugs comprises determining, based on an application of natural language processing (NLP) to drug description data associated with the plurality of drugs, the drug feature data associated with the plurality of drugs.

18. The method of claim 17, wherein the drug description data associated with the plurality of drugs is received from a public data source.

19. The method of claim 16, wherein the drug feature data comprise one or more of drug color, drug size, drug flavor, drug shape, drug manufacturer, therapeutic class, active ingredients of a drug, or inactive ingredients of a drug.

20. The method of claim 16, wherein the at least one substitution drug is associated with the drug feature data.
